(19) United States
(12) Patent Application Publication
(10) Pub. No.: US 2022/0233404 A1
(43) Pub. Date: Jul. 28, 2022

(54) CONTAINERS, COMPOSITIONS, AND METHODS RELATED TO CANNABINOID ANIONS

(71) Applicant: Natural Extraction Systems, LLC, Boulder, CO (US)

(72) Inventors: C. Russell Thomas, Boulder, CO (US); Douglas G. Metcalf, Boulder, CO (US)

(21) Appl. No.: 17/621,879

(22) PCT Filed: Jun. 25, 2020
(86) PCT No.: PCT/US20/39672

§ 371 Date: Dec. 22, 2021

Related U.S. Application Data
(60) Provisional application No. 62/867,209, filed on Jun. 26, 2019.
(60) Provisional application No. 62/928,946, filed on Oct. 31, 2019.
(60) Provisional application No. 62/935,486, filed on Nov. 14, 2019.

(51) Int. Cl. A61J 1/20; A61K 31/05; A61J 1/14

(52) U.S. Cl. CPC A61J 1/2093; A61J 1/1418; A61J 1/2027; A61K 31/05

(57) ABSTRACT

Various aspects of this patent document relate cannabinoid anions including methods to produce cannabinoid anions, compositions comprising cannabinoid anions, containers that contain compositions comprising cannabinoid anions, and methods to consume cannabinoid anions.

### CROSS-REFERENCE TO RELATED APPLICATIONS

This patent application claims priority to U.S. Provisional Patent Application No. 62/867,209, filed Jun. 26, 2019; U.S. Provisional Patent Application No. 62/928,946, filed Oct. 31, 2019; and U.S. Provisional Patent Application No. 62/935,486, filed Nov. 14, 2019, each of which is incorporated by reference in its entirety.

### BACKGROUND

Cannabinoids are generally insoluble in water. Emulsification is generally used to produce cannabinoid beverages by suspending cannabinoids in water. Emulsions frequently display unfavorable characteristics, however, including undesirable flavor and poor bioavailability. Methods of dissolving cannabinoids in water could disrupt the cannabinoid beverage industry.

### SUMMARY

Various aspects of this patent document relate to containers that contain cannabinoid anions that dissolve in water. Cannabinoid anions convert into cannabinoid molecules at neutral or acidic pH, which reduces the compatibility of cannabinoid anions with many ingredients. Reconverted neutrally-charged cannabinoids molecules can nevertheless remain dissolved or suspended in water for minutes-to-hours, which improves their bioavailability relative to historical cannabinoid beverage formulations. Some aspects of this patent document relate to containers that protect cannabinoid anions from neutral or acidic pH and nevertheless allow them to mix with incompatible ingredients minutes-to-hours prior to consumption.

### BRIEF DESCRIPTION OF THE DRAWINGS

The drawings depict specific embodiments that fall within the scope of various claims, and the drawings shall not be used to limit the scope of any claim.

FIG. 1A-1H are diagrams of containers 1 that each contain a composition 2 (shaded regions) and a second composition (circumscribed white regions within each container) that are separated by a barrier (black lines that separate shaded regions from circumscribed white regions within each container) that inhibits fluid communication between each composition and each second composition. Each container also includes an opening mechanism 3 that is configured to open the container.

FIG. 2A is a diagram of a container 1 that includes a barrier (black line that separates the shaded region from the circumscribed white regions within the container) that inhibits fluid communication between a composition 2 (shaded region) and a second composition (circumscribed white regions within the container). The container includes an opening mechanism 3 that is in mechanical communication with an actuator (black isosceles trapezoid). The actuator is in a first state that inhibits fluid communication between the composition and the second composition.

FIG. 2B is a diagram of the same container as in FIG. 2A after the opening mechanism has been used to open the container. The actuator is in a second state that allows fluid communication between the composition and the second composition.

FIG. 2C is a diagram of the same container as in FIG. 2A and FIG. 2B after the creation of fluid communication between the composition and the second composition.

FIG. 3A is a diagram of a container 1 that includes a barrier (black line that separates the shaded region from the lower circumscribed white region) that inhibits fluid communication between a composition 2 (shaded region) and a second composition (lower circumscribed white region). The container includes an opening mechanism 3 that is configured to open the container

FIG. 3B is a diagram of the same container as in FIG. 3A after the creation of fluid communication between the composition and the second composition.

FIG. 3C is a diagram of the same container as in FIG. 3A and FIG. 3B after the opening mechanism has been used to open the container.

FIG. 4A is a diagram of a container 1 that includes a barrier (black line that separates the shaded region from the lower circumscribed white region) that inhibits fluid communication between a composition 2 (shaded region) and a second composition (lower circumscribed white region). The container includes an opening mechanism 3 that is configured to open the container.

FIG. 4B is a diagram of the same container as in FIG. 4A after the creation of fluid communication between the composition and the second composition.

FIG. 4C is a diagram of the same container as in FIG. 4A and FIG. 4B after the opening mechanism has been used to open the container.

FIG. 5A is a diagram of a container 1 that includes a barrier (black lines that separate the shaded region from the two lower circumscribed white regions) that inhibits fluid communication between a composition 2 (shaded region) and a second composition (two lower circumscribed white regions). The container includes an opening mechanism 3 that is configured to open the container.

FIG. 5B is a diagram of the same container as in FIG. 5A after the opening mechanism has been used to open the container.

FIG. 5C is a diagram of the same container as in FIG. 5A and FIG. 5B after the creation of fluid communication between the composition and the second composition.

FIG. 6A is a diagram of a container 1 that includes a barrier (black lines that separate the shaded region from the two lower circumscribed white regions) that inhibits fluid communication between a composition 2 (shaded region) and a second composition (two lower circumscribed white regions). The barrier comprises an actuator that is in mechanical communication with a push-button. The push-button is in an un-pushed state, and the two arrows depict where the push-button may be pushed to change the push-button to a pushed state. The actuator is in a first state that inhibits fluid communication between the composition and the second composition. The container also includes an opening mechanism 3 that is configured to open the container.

FIG. 6B is a diagram of the same container as in FIG. 6A after the push-button has been pushed to change the push-button to the pushed state. The actuator is in a second state that allows fluid communication between the composition and the second composition.

FIG. 6C is a diagram of the same container as in FIG. 6A and FIG. 6B after the creation of fluid communication between the composition and the second composition.

FIG. 7A is a diagram of a container 1 that includes a barrier (black line that separates the shaded region from the lower circumscribed white region) that inhibits fluid communication between a composition 2 (shaded region) and a second composition (lower circumscribed white region). The container includes an opening mechanism 3 that is configured to open the container.

FIG. 7B is a diagram of the same container as in FIG. 7A after the opening mechanism has been used to open the container.

FIG. 7C is a diagram of the same container as in FIG. 7A and FIG. 7B after the creation of fluid communication between the composition and the second composition.

FIG. 8A is a diagram of a container 1 that includes a barrier (black line that separates the shaded region from the lower circumscribed white region) that inhibits fluid communication between a composition 2 (shaded region) and a second composition (lower circumscribed white region). The barrier includes a labile region (circle). The container includes an opening mechanism 3 that is configured to open the container.

FIG. 8B is a diagram of the same container as in FIG. 8A, in which a chemical reaction has permeabilized the labile region (semicircle).

FIG. 8C is a diagram of the same container as in FIG. 8A and FIG. 8B after the opening mechanism has been used to open the container. The composition has a variable pressure, and the opening of the container has changed the variable pressure to create fluid communication between the composition and the second composition.

FIG. 8D is a diagram of the same container as in FIG. 8A, FIG. 8B, and FIG. 8C after the creation of fluid communication between the composition and the second composition.

FIG. 9A is a diagram of a container 1 that includes a barrier (black line that separates the shaded region from the lower circumscribed white region) that inhibits fluid communication between a composition 2 (shaded region) and a second composition (lower circumscribed white region). The barrier comprises an actuator that is in mechanical communication with a push-button. The push-button is in an un-pushed state, and the arrow depicts where the push-button may be pushed to change the push-button to a pushed state. The actuator is in a first state that inhibits fluid communication between the composition and the second composition. The container also includes an opening mechanism 3 that is configured to open the container.

FIG. 9B is a diagram of the same container as in FIG. 9A after the push-button has been pushed to change the push-button to the pushed state. The actuator is in a second state that allows fluid communication between the composition and the second composition.

FIG. 9C is a diagram of the same container as in FIG. 9A and FIG. 9B after the creation of fluid communication between the composition and the second composition.

FIG. 10A is a diagram of a container 1 and a second container 3, in which the second container contains a composition 2, and the container contains a second composition (large white region).

FIG. 10B is a diagram of the same container and second container as in FIG. 10A after an opening mechanism has been used to open the second container.

FIG. 10C is a diagram of the same container and second container as in FIG. 10A and FIG. 10B after the creation of fluid communication between the composition and the second composition.

FIG. 11A is a diagram of a container 1 and a second container 2, in which the second container contains a composition (shaded region), and the container contains a second composition (large white region).

FIG. 11B is a diagram of the same container and second container as in FIG. 11A after the second container has been placed in the container such that (i) the container now contains the composition and (ii) the second container is a barrier that inhibits fluid communication between the composition and the second composition.

FIG. 11C is a diagram of the same container and second container as in FIG. 11A and FIG. 11B after a chemical reaction has created fluid communication between the composition and the second composition

FIG. 12A is a diagram of a container 1 and a second container 2, in which the second container contains a composition (shaded region), and the container contains a second composition (large white region).

FIG. 12B is a diagram of the same container and second container as in FIG. 12A after the second container has been placed in the container such that the container now contains the composition.

FIG. 12C is a diagram of the same container and second container as in FIG. 12A and FIG. 12B after the creation of fluid communication between the composition and the second composition.

FIG. 13 is a diagram of a container that contains a composition 1 (shaded region), a second composition 2 (white region), a barrier that inhibits fluid communication between the composition and the second composition (vertical lines that separate the shaded region from the white region), and a mouthpiece 3.

FIG. 14 is a diagram of a container that contains a composition 1 (shaded region), a second composition 2 (white region), a barrier that inhibits fluid communication between the composition and the second composition (vertical lines that separate the shaded region from the second composition), a mouthpiece 3, and a propellant 4 (white region).

FIG. 15 is a diagram of a container that contains a composition 1 (shaded region), a second composition 2 (white region), a barrier that inhibits fluid communication between the composition and the second composition (vertical lines that separate the shaded region from the white region), a mouthpiece 3, and a spray nozzle 5.

FIG. 16 is a diagram of a container that contains a composition 1 (shaded region), a second composition 2 (white region), a barrier that inhibits fluid communication between the composition and the second composition (vertical lines that separate the shaded region from the second composition), a mouthpiece 3, a propellant 4 (white region), and a spray nozzle 5.

FIG. 17 is a diagram of a container that contains a composition 1 (shaded region), a second composition 2 (white region), a barrier that inhibits fluid communication between the composition and the second composition (horizontal line that separates the shaded region from the white region), a mouthpiece 3, and a mixing chamber 6 for creating fluid communication between the composition and the second composition.

FIG. 18 is a diagram of a container that contains a composition 1 (shaded region), a second composition 2 (white region), a barrier that inhibits fluid communication between the composition and the second composition (horizontal line that separates the shaded region from the white region), a mouthpiece 3, a spray nozzle 5, and a mixing chamber 6 for creating fluid communication between the composition and the second composition.

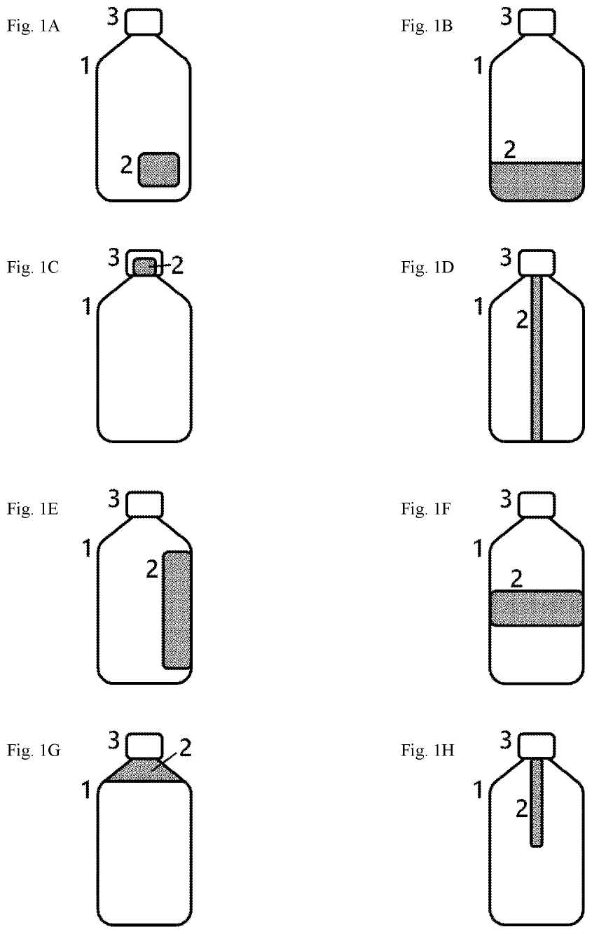

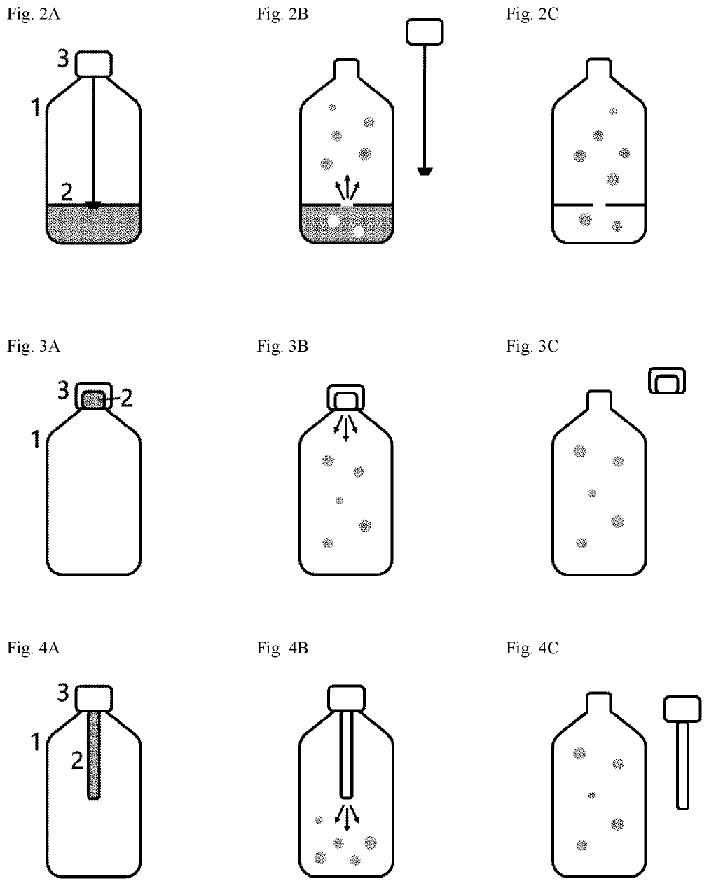

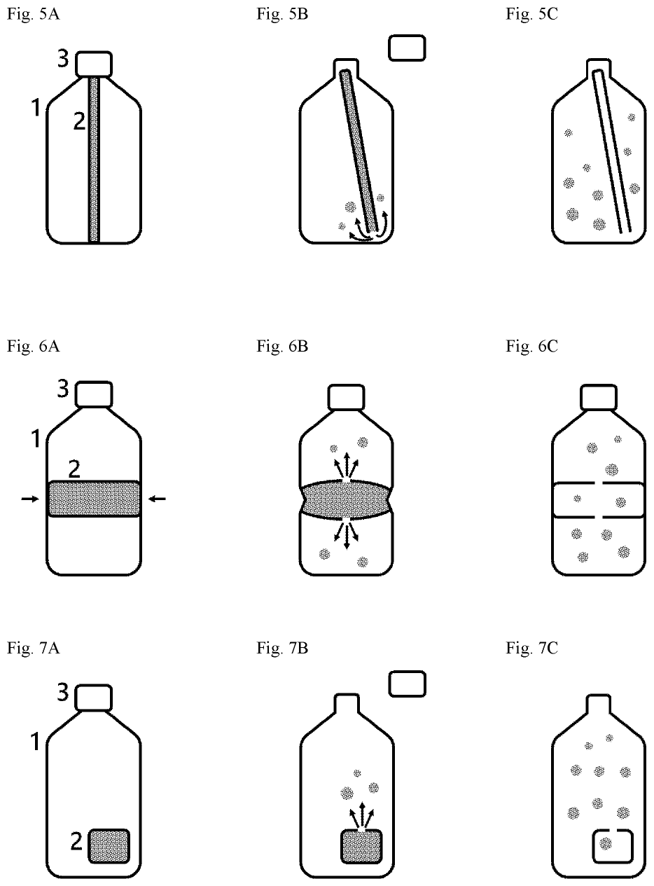

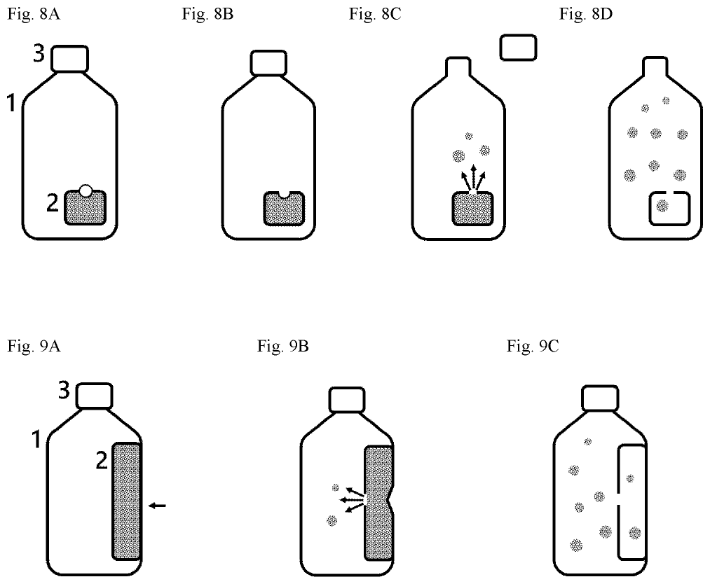

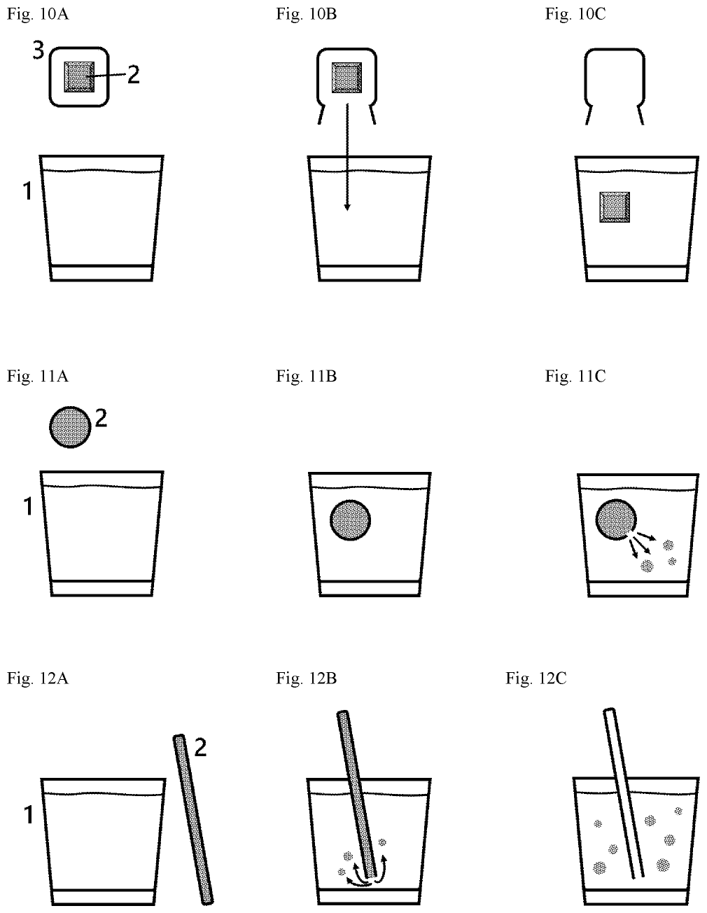

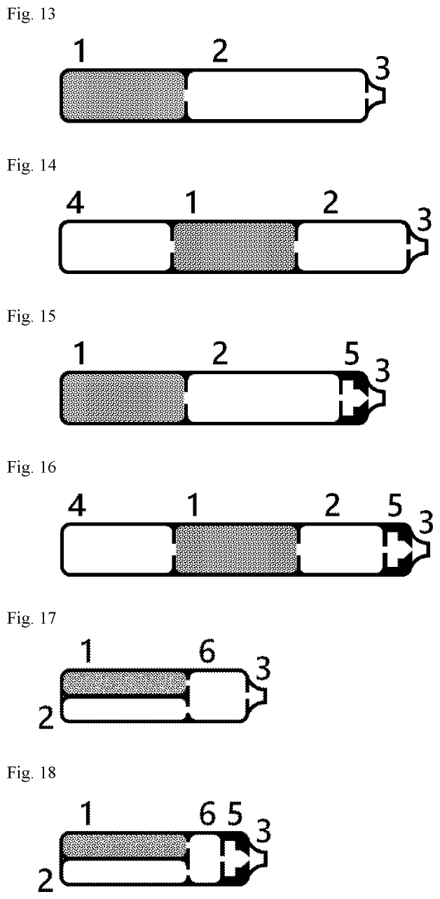

### DETAILED DESCRIPTION

Various aspects of this patent document relate to a method to solubilize a cannabinoid in water, comprising: (1) providing a cannabinoid molecule, the cannabinoid molecule is cannabidiol and comprises an aromatic ring and a hydroxyl group, and the hydroxyl group is a substituent on the aromatic ring; (2) providing a Brønsted base and ethanol; (3) providing water; (4) contacting the cannabinoid molecule with the Brønsted base and the ethanol to deprotonate the hydroxyl group and to produce an anionic cannabinoid molecule; and (5) dissolving the anionic cannabinoid molecule in the water to produce a solution comprising the anionic cannabinoid molecule, in which the solution comprising the anionic cannabinoid molecule has a pH of at least 8.5; and the anionic cannabinoid molecule is selected from the group consisting of: 2-[(1R,6R)-6-isopropenyl-3-methylcyclohex-2-en-1-yl]-3-hydroxy-5-pentylphenolate; 2-[(1R,6R)-6-isopropenyl-3-methylcyclohex-3-en-1-yl]-3-hydroxy-5-pentylphenolate; 2-[(1R,6R)-6-isopropenyl-3-methylcyclohex-2-en-1-yl]-5-pentyl-1,4-benzoquinone-3-oxide; 3-[(1R,6R)-6-isopropenyl-3-methylcyclohex-2-en-1-yl]-6-pentyl-1,2-benzoquinone-4-oxide; 2-[(1R,6R)-6-isopropenyl-3-methylcyclohex-3-en-1-yl]-5-pentyl-1,4-benzoquinone-3-oxide; 3-[(1R,6R)-6-isopropenyl-3-methylcyclohex-3-en-1-yl]-6-pentyl-1,2-benzoquinone-4-oxide; and (6aR,10aR)-6,6,9-trimethyl-3-pentyl-6a,7,10,10a-tetrahydro-6H-benzo[c]chromen-1-oxide.

The term “dissolved” refers to a chemical species that is a solute of a solvent such as water. A chemical species that is merely suspended within a solvent, such as a molecule of an emulsion, is not dissolved. A chemical species that exists in a complex with a dissolved solute is not dissolved. A cannabinoid that is covalently or non-covalently associated with another cannabinoid, another lipid, an amphiphilic molecule, a carbohydrate (such as a cyclodextrin), or a polymer (such as polyethylene glycol) is not dissolved in a solvent.

Various aspects of this patent document relate to a method of consuming a cannabinoid, comprising: (1) providing a composition comprising an anionic cannabinoid molecule dissolved in water, in which the composition has a color; (2) contacting the composition with a Brønsted acid, in which contacting the composition with the Brønsted acid changes the color to either a different color or no color; and (3) consuming the composition after contacting the composition with the Brønsted acid, in which a human being consumes the composition by drinking it, wherein the anionic cannabinoid molecule is selected from any cannabinoid anion as defined below. In some embodiments, the different color is yellow. “No color” is synonymous with “colorless.”

Various aspects of this patent document relate to a method of consuming a cannabinoid, comprising: (1) providing a hermetically-sealed container that contains a composition, in which: the hermetically-sealed container is a glass bottle, plastic bottle, or aluminum can; the container contains 25 milliliters to 800 milliliters of the composition; the composition comprises water and 50 micrograms to 500 milligrams of an anionic cannabinoid molecule; the anionic cannabinoid molecule is dissolved in the water; and the composition has a color; (2) unsealing the container; (3) contacting the composition with a Brønsted acid, in which contacting the composition with the Brønsted acid changes the color to either a different color or no color; and (4) consuming the composition after contacting the composition with the Brønsted acid, in which a human being consumes the composition by drinking it, wherein the anionic cannabinoid molecule is selected from any cannabinoid anion as defined below. In some embodiments, the different color is yellow.

Various aspects of this patent document relate to a method to change the color of a composition, comprising: (1) providing a container that contains a composition, in which: the composition comprises an anionic cannabinoid molecule dissolved in water; the anionic cannabinoid molecule is 2-[(1R,6R)-6-isopropenyl-3-methylcyclohex-2-en-1-yl]-3-hydroxy-5-pentylphenolate; the composition has a color; and the color is purple; and (2) contacting the composition with a Brønsted acid, in which contacting the composition with the Brønsted acid changes the color from purple to either a different color or no color. In some embodiments, the different color is yellow.

Various aspects of this patent document relate to a liquid composition, comprising 2-[(1R,6R)-6-isopropenyl-3-methylcyclohex-2-en-1-yl]-3-hydroxy-5-pentylphenolate, 2-[(1R,6R)-6-isopropenyl-3-methylcyclohex-2-en-1-yl]-5-pentylbenzene-1,3-diol, water, and one or more of sodium ion (Na⁺), potassium ion (K⁺), calcium ion (Ca²⁺), magnesium ion (Mg²⁺), chloride ion (Cl⁻), sulfate (SO₄²⁻), bicarbonate (HCO₃⁻), and carbonate (CO₃²⁻) wherein the 2-[(1R,6R)-6-isopropenyl-3-methylcyclohex-2-en-1-yl]-3-hydroxy-5-pentylphenolate is dissolved in the water; and wherein the liquid composition comprises the 2-[(1R,6R)-6-isopropenyl-3-methylcyclohex-2-en-1-yl]-3-hydroxy-5-pentylphenolate and the 2-[(1R,6R)-6-isopropenyl-3-methylcyclohex-2-en-1-yl]-5-pentylbenzene-1,3-diol at a molar ratio of 1:10 to 10,000:1.

Various aspects of this patent document relate to a composition, comprising 2-[(1R,6R)-6-isopropenyl-3-methylcyclohex-2-en-1-yl]-3-hydroxy-5-pentylphenolate and 2-[(1R,6R)-6-isopropenyl-3-methylcyclohex-2-en-1-yl]-5-pentylbenzene-1,3-diol at a molar ratio of 1:10 to 10,000:1, and further comprising one or more of sodium ion (Na⁺), potassium ion (K⁺), calcium ion (Ca²⁺), magnesium ion (Mg²⁺), chloride ion (Cl⁻), sulfate (SO₄²⁻), bicarbonate (HCO₃⁻), and carbonate (CO₃²⁻).

Various aspects of this patent document relate to a composition, comprising 2-[(1R,6R)-6-isopropenyl-3-methylcyclohex-2-en-1-yl]-3-hydroxy-5-pentylphenolate and 2-[(1R,6R)-6-isopropenyl-3-methylcyclohex-2-en-1-yl]-5-pentylbenzene-1,3-diol at a molar ratio of 1:10 to 10,000:1, wherein the composition comprises a solid phase, the solid phase comprises a salt, and the salt comprises 2-[(1R,6R)-6-isopropenyl-3-methylcyclohex-2-en-1-yl]-3-hydroxy-5-pentylphenolate.

Various aspects of this patent document relate to a composition, comprising 2-[(1R,6R)-6-isopropenyl-3-methylcyclohex-2-en-1-yl]-3-hydroxy-5-pentylphenolate and 2-[(1R,6R)-6-isopropenyl-3-methylcyclohex-2-en-1-yl]-5-pentylbenzene-1,3-diol at a molar ratio of 10,000:1 to 1,000,000:1, and further comprising one or more of sodium ion (Na⁺), potassium ion (K⁺), calcium ion (Ca²⁺), magnesium ion (Mg²⁺), chloride ion (Cl⁻), sulfate (SO₄²⁻), bicarbonate (HCO₃⁻), and carbonate (CO₃²⁻).

Various aspects of this patent document relate to a composition, comprising: 2-[(1R,6R)-6-isopropenyl-3-methylcyclohex-2-en-1-yl]-3-hydroxy-5-pentylphenolate; 2-[(1R,6R)-6-isopropenyl-3-methylcyclohex-2-en-1-yl]-5-pentylbenzene-1,3-diol; water; and potassium ion (K⁺), wherein: the composition is a liquid; and the composition comprises the 2-[(1R,6R)-6-isopropenyl-3-methylcyclohex-2-en-1-yl]-3-hydroxy-5-pentylphenolate and the 2-[(1R,6R)-6-isopropenyl-3-methylcyclohex-2-en-1-yl]-5-pentylbenzene-1,3-diol at a molar ratio of at least 1:10 and no greater than 1,000,000:1.

Various aspects of this patent document relate to a liquid composition, comprising: (1) 2-[(1R,6R)-6-isopropenyl-3-methylcyclohex-2-en-1-yl]-3-hydroxy-5-pentylphenolate at a concentration by weight of at least 5 percent and no greater than 25 percent; (2) 2-[(1R,6R)-6-isopropenyl-3-methylcyclohex-2-en-1-yl]-5-pentylbenzene-1,3-diol, wherein the composition comprises the 2-[(1R,6R)-6-isopropenyl-3-methylcyclohex-2-en-1-yl]-3-hydroxy-5-pentylphenolate and the 2-[(1R,6R)-6-isopropenyl-3-methylcyclohex-2-en-1-yl]-5-pentylbenzene-1,3-diol at a molar ratio of at least 1:10 and no greater than 1,000,000:1; (3) ethanol at a concentration by weight of at least 10 percent and no greater than 95 percent; (4) water at a concentration by weight of at least 1 percent and no greater than 10 percent; and (5) potassium ion (K⁺) at a concentration of at least 10 millimoles per liter and no greater than 1 mole per liter.

Various aspects of this patent document relate to a liquid composition, comprising: (1) 2-[(1R,6R)-6-isopropenyl-3-methylcyclohex-2-en-1-yl]-3-hydroxy-5-pentylphenolate at a concentration by weight of at least 5 parts per million and no greater than 10 percent; (2) 2-[(1R,6R)-6-isopropenyl-3-methylcyclohex-2-en-1-yl]-5-pentylbenzene-1,3-diol, wherein the composition comprises the 2-[(1R,6R)-6-isopropenyl-3-methylcyclohex-2-en-1-yl]-3-hydroxy-5-pentylphenolate and the 2-[(1R,6R)-6-isopropenyl-3-methylcyclohex-2-en-1-yl]-5-pentylbenzene-1,3-diol at a molar ratio of at least 1:1 and no greater than 10,000:1; (3) water; (4) ethanol; and (5) potassium ion (K⁺) and sodium ion (Na⁺) at a combined concentration of at least 10 milligrams per liter and no greater than 1000 milligrams per liter.

Various aspects of this patent document relate to a container, containing at least 80 microliters and no greater than 4 liters of a composition that comprises (i) a solvent, (ii) a cannabinoid anion, and (iii) a cation, wherein: (1) the composition comprises a liquid phase; (2) the liquid phase comprises the solvent at a concentration of at least 5 molar and no greater than 55.5 molar; (3) the liquid phase comprises the cation at a concentration of at least 8 micromolar and no greater than 0.8 molar; (4) the solvent is either water, ethanol, propylene glycol, propane-1,3-diol, or a sugar alcohol; and (5) the cannabinoid anion and the cation are solutes that are dissolved in the solvent.

In some embodiments, the container further contains a second composition that comprises a Brønsted acid, wherein the container comprises a barrier that inhibits fluid communication between the composition and the second composition.

In some embodiments, the composition has a pH of at least 8 and less than 14, and the second composition has a pH of greater than 0 and less than 8. In some specific embodiments, the composition has a pH of at least 9 and no greater than 12, and the second composition has a pH of at least 1 and no greater than 6.

In some embodiments, the second composition comprises water at a concentration of at least 45.5 molar and no greater than 55.5 molar; and the Brønsted acid is dissolved in the water.

In some embodiments, the Brønsted acid is citric acid, carbonic acid, or hydronium.

In some embodiments, the container is configured to create fluid communication between the composition and the second composition.

In some embodiments, the container is configured such that opening the container permeabilizes the barrier to either one or both of the composition and the second composition to create fluid communication between the composition and the second composition.

In some embodiments, the container has structural integrity; and the container is configured such that an appropriate mechanical force can create fluid communication between the composition and the second composition without compromising the structural integrity of the container. “Structural integrity” is the ability of a container to contain both a composition and a second composition. In some embodiments, the container is physically associated with a label, and the label comprises instructions that describe how to apply the appropriate mechanical force.

In some embodiments, the container has structural integrity; and the container is configured such that a chemical reaction can create fluid communication between the composition and the second composition without compromising the structural integrity of the container.

In some embodiments, the container has structural integrity; either the composition has a variable pressure, the second composition has a variable pressure, or both the composition and the second composition have a variable pressure; and the container is configured such that a change in the variable pressure can create fluid communication between the composition and the second composition without compromising the structural integrity of the container.

In some embodiments, the container comprises an opening mechanism that is configured to open the container. In some embodiments, the opening mechanism is a screw cap, a crown cap, a cork, a push tab, or a pull tab. In some embodiments, the container is configured to create fluid communication between the composition and the second composition when the opening mechanism is used to open the container.

In some embodiments, the container is configured to create fluid communication between the composition and the second composition when the opening mechanism is translated in three-dimensional space relative to the rest of the container.

In some embodiments, the opening mechanism is a screw cap; and the container is configured to create fluid communication between the composition and the second composition when the screw cap is rotated in three-dimensional space relative to the rest of the container.

In some embodiments, the container further comprises an actuator. An “actuator” is any feature that has both (1) a first state that inhibits fluid communication between the composition and the second composition; and (2) a second state that allows fluid communication between the composition and the second composition. In some embodiments, the actuator is in mechanical communication with the barrier. In some embodiments, the barrier comprises the actuator.

In some embodiments, the opening mechanism is a screw cap; the screw cap is in mechanical communication with the actuator; and rotating the screw cap in three-dimensional space relative to the rest of the container is operable to change the actuator from the first state to the second state.

In some embodiments, the opening mechanism is in mechanical communication with the actuator; and translating the opening mechanism in three-dimensional space relative to the rest of the container is operable to change the actuator from the first state to the second state.

In some embodiments, the container further comprises a push-button, wherein the actuator is in mechanical communication with the push-button. A “push-button” is any feature that has both (1) an un-pushed state and (2) a pushed state, in which a translation of the push-button (a “push”) in the un-pushed state is operable to change the push-button from its un-pushed state to its pushed state. In some embodiments, the push-button is a button. In some embodiments, the push-button is a wall, panel, or region of the container. In some embodiments, the push-button is in the un-pushed state; pushing the push-button in the un-pushed state is operable to change the push-button from its un-pushed state to its pushed state; and changing the push-button from its un-pushed state to its pushed state is operable to change the actuator from its first state to its second state.

In some embodiments, the actuator inhibits the opening mechanism from opening the container in its first state; and the actuator allows the opening mechanism to open the container in its second state.

In some embodiments, the composition has a color, and fluid communication between the composition and the second composition is operable to change the color of the composition either to a different color or to colorless. In some embodiments, the different color is yellow.

In some embodiments, the composition has a color, and the color is either a shade of purple, a shade of red, or a shade of brown. In some specific embodiments, the color is purple. In specific some embodiments, the color is red. In specific some embodiments, the color is maroon. In specific some embodiments, the color is brown.

In some embodiments, the liquid phase has a pH of at least 8.5 and no greater than 14. In some specific embodiments, the liquid phase has a pH of at least 9 and no greater than 12. In some very specific embodiments, the liquid phase has a pH of at least 9.5 and no greater than 11.5.

In some embodiments, the solvent is water. In some specific embodiments, the solvent is water, and the composition comprises glycerol. In some specific embodiments, the solvent is water, and the composition comprises ethanol. In some very specific embodiments, the solvent is water, and the composition comprises both glycerol and ethanol.

In some embodiments, the solvent is a sugar alcohol, and the sugar alcohol is either glycerol, erythritol, xylitol, mannitol, sorbitol, or inositol. In some specific embodiments, the solvent is a sugar alcohol, and the composition comprises ethanol. In some very specific embodiments, the solvent is a sugar alcohol, and the composition comprises both ethanol and water.

In some embodiments, the solvent is glycerol. In some specific embodiments, the solvent is a glycerol, and the composition comprises either ethanol, water, or both ethanol and water.

In some embodiments, the solvent is ethanol. In some specific embodiments, the solvent is ethanol, and the composition comprises water.

In some embodiments, the cation is either ammonium (“NH₄+”); protonated ethanolamine; choline; protonated sphingosine; protonated lysine; or protonated arginine.

In some embodiments, the cation is either sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”). In some specific embodiments, the cation is sodium cation. In some specific embodiments, the cation is potassium cation.

A “cannabinoid anion” is a cannabinoid that both (1) carries a net negative charge and (2) lacks a carboxyl group. Cannabinoid anions include, but are not limited to, cannabinoids that have a deprotonated hydroxyl oxygen such that either (i) the cannabinoid anion contains an oxide, such as a phenolate, or (ii) a resonance structure of the cannabinoid anion contains an oxide, such as a phenolate. U.S. Pat. No. 10,555,914 B1 describes methods of producing “anionic cannabinoid molecules,” which is synonymous with “cannabinoid anions,” and PCT Patent Application Publication No. WO 2020/123809 A1 names various cannabinoid anions. This document incorporates each of U.S. Pat. No. 10,555,914 B1 and WO 2020/123809 A1 by reference in its entirety to delineate specific and generic anionic cannabinoid molecules that fall within the scope of “cannabinoid anion” as the term is used in the specification and claims.

In all embodiments, the cannabinoid anion is not a carboxylate. In some embodiments, the cannabinoid anion is a phenolate.

In some specific embodiments, the cannabinoid anion is either: 2-[(1R,6R)-6-isopropenyl-3-methylcyclohex-2-en-1-yl]-3-hydroxy-5-pentylphenolate; 2-[(1R,6R)-6-isopropenyl-3-methylcyclohex-3-en-1-yl]-3-hydroxy-5-pentylphenolate; 2-[(1R,6R)-6-isopropenyl-3-methylcyclohex-2-en-1-yl]-5-pentyl-1,4-benzoquinone-3-oxide; 3-[(1R,6R)-6-isopropenyl-3-methylcyclohex-2-en-1-yl]-6-pentyl-1,2-benzoquinone-4-oxide; 2-[(1R,6R)-6-isopropenyl-3-methylcyclohex-3-en-1-yl]-5-pentyl-1,4-benzoquinone-3-oxide; 3-[(1R,6R)-6-isopropenyl-3-methylcyclohex-3-en-1-yl]-6-pentyl-1,2-benzoquinone-4-oxide; (6aR,10aR)-6,6,9-trimethyl-3-pentyl-6a,7,8,10a-tetrahydro-6H-benzo[c]chromen-1-oxide; (6aR,10aR)-6,6,9-trimethyl-3-pentyl-6a,7,10,10a-tetrahydro-6H-benzo[c]chromen-1-oxide; 2-[(2E)-3,7-dimethylocta-2,6-dienyl]-3-hydroxy-5-pentylphenolate; 2-[(1R,6R)-6-isopropenyl-3-methylcyclohex-2-en-1-yl]-3-hydroxy-5-propylphenolate; 2-[(1R,6R)-6-isopropenyl-3-methylcyclohex-3-en-1-yl]-3-hydroxy-5-propylphenolate; 2-[(1R,6R)-6-isopropenyl-3-methylcyclohex-2-en-1-yl]-5-propyl-1,4-benzoquinone-3-oxide; 3-[(1R,6R)-6-isopropenyl-3-methylcyclohex-2-en-1-yl]-6-propyl-1,2-benzoquinone-4-oxide; 2-[(1R,6R)-6-isopropenyl-3-methylcyclohex-3-en-1-yl]-5-propyl-1,4-benzoquinone-3-oxide; 3-[(1R,6R)-6-isopropenyl-3-methylcyclohex-3-en-1-yl]-6-propyl-1,2-benzoquinone-4-oxide; (6aR,10aR)-6,6,9-trimethyl-3-propyl-6a,7,8,10a-tetrahydro-6H-benzo[c]chromen-1-oxide; (6aR,10aR)-6,6,9-trimethyl-3-propyl-6a,7,10,10a-tetrahydro-6H-benzo[c]chromen-1-oxide; or 2-[(2E)-3,7-dimethylocta-2,6-dienyl]-3-hydroxy-5-propylphenolate.

In some very specific embodiments, the cannabinoid anion is 2-[(1R,6R)-6-isopropenyl-3-methylcyclohex-2-en-1-yl]-3-hydroxy-5-pentylphenolate.

In some very specific embodiments, the cannabinoid anion is (6aR,10aR)-6,6,9-trimethyl-3-pentyl-6a,7,8,10a-tetrahydro-6H-benzo[c]chromen-1-oxide.

In some very specific embodiments, the cannabinoid anion is 2-[(2E)-3,7-dimethylocta-2,6-dienyl]-3-hydroxy-5-pentylphenolate.

In some very specific embodiments, the cannabinoid anion is 2-[(1R,6R)-6-isopropenyl-3-methylcyclohex-2-en-1-yl]-3-hydroxy-5-propylphenolate.

In some very specific embodiments, the cannabinoid anion is (6aR,10aR)-6,6,9-trimethyl-3-propyl-6a,7,8,10a-tetrahydro-6H-benzo[c]chromen-1-oxide.

In some very specific embodiments, the cannabinoid anion is 2-[(2E)-3,7-dimethylocta-2,6-dienyl]-3-hydroxy-5-propylphenolate.

In some embodiments, the container is either a cup, a glass, a mug, a bottle, a can, or a jug.

In some embodiments, the container is either a vaporizer, an atomizer, a nebulizer, or an electronic cigarette.

In some embodiments, the container is a cartridge configured for use with either a vaporizer, an atomizer, a nebulizer, or an electronic cigarette.

In some embodiments, the container is packaging.

In some embodiments, the container is hermetically-sealed.

1-6. (canceled)

7. A composition, comprising 2-[(1R,6R)-6-isopropenyl-3-methylcyclohex-2-en-1-yl]-3-hydroxy-5-pentylphenolate and 2-[(1R,6R)-6-isopropenyl-3-methylcyclohex-2-en-1-yl]-5-pentylbenzene-1,3-diol at a molar ratio of 1:10 to 10,000:1, wherein the composition comprises a solid phase, the solid phase comprises a salt, and the salt comprises 2-[(1R,6R)-6-isopropenyl-3-methylcyclohex-2-en-1-yl]-3-hydroxy-5-pentylphenolate.

8. A composition, comprising 2-[(1R,6R)-6-isopropenyl-3-methylcyclohex-2-en-1-yl]-3-hydroxy-5-pentylphenolate and 2-[(1R,6R)-6-isopropenyl-3-methylcyclohex-2-en-1-yl]-5-pentylbenzene-1,3-diol at a molar ratio of 10,000:1 to 1,000,000:1, and further comprising one or more of sodium ion (Na⁺), potassium ion (K⁺), calcium ion (Ca²⁺), magnesium ion (Mg²⁺), chloride ion (Cl⁻), sulfate (SO₄²⁻), bicarbonate (HCO₃⁻), and carbonate (CO₃²⁻).

9-11. (canceled)

12. A container, containing at least 80 microliters and no greater than 4 liters of a composition that comprises (i) a solvent, (ii) a cannabinoid anion, and (iii) a cation; and containing a second composition that comprises a Brønsted acid, wherein:
the composition comprises a liquid phase;
the liquid phase comprises the solvent at a concentration of at least 5 molar and no greater than 55.5 molar;
the liquid phase comprises the cation at a concentration of at least 8 micromolar and no greater than 0.8 molar;
the solvent is either water, ethanol, propylene glycol, propane-1,3-diol, or a sugar alcohol;
the cannabinoid anion and the cation are solutes that are dissolved in the solvent; and
the container comprises a barrier that inhibits fluid communication between the composition and the second composition.

13. (canceled)

14. The container of claim 12, wherein the composition has a pH of at least 8 and less than 14, and the second composition has a pH of greater than 0 and less than 8.

15. The container of claim 12, wherein the second composition comprises water at a concentration of at least 45.5 molar and no greater than 55.5 molar; and the Brønsted acid is dissolved in the water.

16. (canceled)

17. The container of claim 12, wherein the container is configured to create fluid communication between the composition and the second composition.

18. (canceled)

19. The container of claim 17, wherein the container has structural integrity, which is the ability of the container to contain both the composition and the second composition; and the container is configured such that an appropriate mechanical force can create fluid communication between the composition and the second composition without compromising the structural integrity of the container.

20-22. (canceled)

23. The container of claim 12, wherein the container comprises an opening mechanism that is configured to open the container; and the container is configured to create fluid communication between the composition and the second composition when the opening mechanism is used to open the container.

24-25. (canceled)

26. The container of claim 23, wherein the container is configured to create fluid communication between the composition and the second composition when the opening mechanism is translated in three-dimensional space relative to the rest of the container.

27. The container of claim 23, wherein the opening mechanism is a screw cap; and the container is configured to create fluid communication between the composition and the second composition when the screw cap is rotated in three-dimensional space relative to the rest of the container.

28. The container claim 23, further comprising an actuator, wherein the actuator has a first state and a second state; the actuator is in the first state; the first state inhibits fluid communication between the composition and the second composition; and the second state allows fluid communication between the composition and the second composition.

29. The container of claim 28, wherein the opening mechanism is in mechanical communication with the actuator; and translating the opening mechanism in three-dimensional space relative to the rest of the container is operable to change the actuator from the first state to the second state.

30. The container of claim 28, wherein the opening mechanism is a screw cap; the screw cap is in mechanical communication with the actuator; and rotating the screw cap in three-dimensional space relative to the rest of the container is operable to change the actuator from the first state to the second state.

31. The container of claim 28, further comprising a push-button, wherein the actuator is in mechanical communication with the push-button; the push-button has an un-pushed state and a pushed state; the push-button is in the un-pushed state; pushing the push-button in the un-pushed state is operable to change the push-button from its un-pushed state to its pushed state; and changing the push-button from its un-pushed state to its pushed state is operable to change the actuator from its first state to its second state.

32. (canceled)

33. The container of claim 12, wherein the composition has a color, and fluid communication between the composition and the second composition is operable to change the color of the composition either to a different color or to colorless.

34. The container of claim 33, wherein the color of the composition is either a shade of purple, a shade of red, or a shade of brown.

35-39. (canceled)

40. The container of claim 12, wherein the solvent is a sugar alcohol.

41-43. (canceled)

44. The container of claim 12, wherein the cation is either sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”).

45-48. (canceled)

49. The container of claim 12, wherein the cannabinoid anion is either:
2-[(1R,6R)-6-isopropenyl-3-methylcyclohex-2-en-1-yl]-3-hydroxy-5-pentylphenolate;
2-[(1R,6R)-6-isopropenyl-3-methylcyclohex-3-en-1-yl]-3-hydroxy-5-pentylphenolate;
2-[(1R,6R)-6-isopropenyl-3-methylcyclohex-2-en-1-yl]-5-pentyl-1,4-benzoquinone-3-oxide;
3-[(1R,6R)-6-isopropenyl-3-methylcyclohex-2-en-1-yl]-6-pentyl-1,2-benzoquinone-4-oxide;
2-[(1R,6R)-6-isopropenyl-3-methylcyclohex-3-en-1-yl]-5-pentyl-1,4-benzoquinone-3-oxide;
3-[(1R,6R)-6-isopropenyl-3-methylcyclohex-3-en-1-yl]-6-pentyl-1,2-benzoquinone-4-oxide;
2-[(1R,6R)-6-isopropenyl-3-methylcyclohex-2-en-1-yl]-3-hydroxy-5-propylphenolate;
2-[(1R,6R)-6-isopropenyl-3-methylcyclohex-3-en-1-yl]-3-hydroxy-5-propylphenolate;
2-[(1R,6R)-6-isopropenyl-3-methylcyclohex-2-en-1-yl]-5-propyl-1,4-benzoquinone-3-oxide;
3-[(1R,6R)-6-isopropenyl-3-methylcyclohex-2-en-1-yl]-6-propyl-1,2-benzoquinone-4-oxide;
2-[(1R,6R)-6-isopropenyl-3-methylcyclohex-3-en-1-yl]-5-propyl-1,4-benzoquinone-3-oxide; or
3-[(1R,6R)-6-isopropenyl-3-methylcyclohex-3-en-1-yl]-6-propyl-1,2-benzoquinone-4-oxide.

50. The container of claim 12, wherein the cannabinoid anion is 2-[(1R,6R)-6-isopropenyl-3-methylcyclohex-2-en-1-yl]-3-hydroxy-5-pentylphenolate.

51-60. (canceled)
